(19) United States
(12) Patent Application Publication
(10) Pub. No.: US 2021/0244852 A1
(43) Pub. Date: Aug. 12, 2021

(54) Composition of corneal implantation, and the use and preparation method thereof

(71) Applicant: National Taiwan University, Taipei City (TW)

(72) Inventors: I-JONG WANG, Taipei City (TW); TAI-HORNG YOUNG, Taipei City (TW); I-NI CHIANG, Taipei City (TW); YU-LUN LIU, Taipei City (TW); TING-YUN SHIUE, Taipei City (TW)

(21) Appl. No.: 17/105,587

(22) Filed: Nov. 26, 2020

(30) Foreign Application Priority Data
Feb. 12, 2020 (TW) 109104372

(51) Int. Cl. A61L 27/24; A61L 27/38; A61L 27/54; A61L 27/50

(52) U.S. Cl. CPC A61L 27/24; A61L 27/3813; A61L 27/3839; A61L 2430/16; A61L 27/50; A61L 27/3895; A61L 2300/64; A61L 27/54

(57) ABSTRACT

The present application provides a composition of corneal implantation, comprising: a collagen film, and renal proximal tubule cells which attached to the collagen film. In addition, the present application further provides a use of the composition of corneal implantation for implanting a patient with damaged cornea endothelium cells and a preparation method of the composition of corneal implantation.

### CROSS-REFERENCE TO RELATED APPLICATIONS

This application claims the priority of Republic of China Patent Application No. 109104372 filed on Feb. 12, 2020, in the State Intellectual Property Office of the R.O.C., the disclosure of which is incorporated herein by reference.

### BACKGROUND OF THE INVENTION

### Field of the Invention

The present invention is a composition of autologous cell membranes, particularly a composition of autologous renal proximal tubule cells cultivated on a collagen film as corneal implantations.

### Descriptions of the Related Art

More than 10 million people in the world are blind due to corneal injury or disease. In 2012, the global market of ophthalmic medical materials reached 34.82 billion U.S. dollars. It is estimated that the market will reach 41.8 billion U.S. dollars in recent years. There are about 200,000 people per year worldwide undergo corneal transplantation, and half of the reason is caused by the loss of corneal endothelial cells.

Corneal Endothelial Cells mainly help the cornea to drain water and have no regeneration function. The number will be reduced due to age, long-term hypoxia, disease, etc. When the number is low to a certain level, it will cause vision influences. Corneal endothelial cells have the function of maintaining the normal water content and transparency of the cornea. The occurrence of lesions will cause corneal swelling and blur and affect vision. In the past, the treatment of corneal endothelial cell abnormalities required full-thickness keratoplasty. Clinically, however, the problems of merely losing corneal endothelial cells, such as pseudophakic bullous keratopathy or primary corneal endothelial cell degeneration diseases, etc., theoretically only need posterior lamellar keratoplasty to replace the diseased corneal endothelium. Like other organ transplantations, corneal transplantations also face a shortage of organ sources.

At present, corneal endothelial transplantation can be used for functional defects of corneal endothelial cells. In Descemet Stripping Automated Endothelial Keratoplasty (DSAEK), the donor's cornea is pre-treated before transplantation. The corneal epithelium and stroma are removed with a knife, leaving parts of the posterior corneal stroma and corneal endothelium, and then transplanted into the patient's eye through a small wound. Because of the wound is much smaller than that of full-thickness corneal transplantation, DSAEK avoids complications caused by corneal astigmatism and corneal sutures. Some documents also point out that the probability of rejection after surgery is lower than that of full-thickness corneal transplantation. However, the thickness of the graft with corneal endothelium and partial posterior corneal stroma that transplanted into the patient's eye is about 150 μm in DSAEK surgery. Sometimes, the recovery of vision may be affected due to the unsmooth cutting of the grafts. So after DSAEK, Descemet's membrane endothelial keratoplasty (DMEK) has been developed.

The thickness of the graft of DMEK transplantation is about 10˜15 μm. It only contains the corneal endothelial layer and Descemet's layer and does not contain the posterior corneal stroma. The postoperative vision recovery of patients is excellent. According to statistics, more than 80% of patients have visual acuity above 0.8 after a successful surgery. Compared with DSAEK, DMEK can provide better visual quality. However, it requires high levels of skill to transplant a graft with a thickness of about 10-15 μm into the patient's eye.

Chiang I-Ni's doctoral dissertation “Tissue Engineering of Renal Proximal Tubule Cell” published in 2017 discussed the culture of renal proximal tubule cells (RPTCs), and disclosed the corneal endothelial cells and the renal proximal tubule cells have the same function of regulating water and both have sodium-potassium pumps and water channels. In the rabbit modules, the autologous renal proximal tubule cells are cultivated on chitosan and organosilicon (PDMS) films and implant for repairing the damaged corneal endothelial cells. One week after implantation, the autologous renal proximal tubule cells are still alive in the eyeball, but the eyeballs of the rabbits appear blurred and swollen after implanting the complex of renal proximal tubule cells/chitosan. While the activities of the human renal proximal tubule cells cultivated on PDMS are not good.

Therefore, there is still a need for a safe, convenient, and easy-to-operate corneal keratoplasty for the treatment of patients with damaged endothelial cells.

### SUMMARY OF THE INVENTION

In view of the problems of the prior art, this application provides a composition of corneal implantation, comprising: a collagen film, and renal proximal tubule cells, attached to the collagen film.

In an embodiment, the density of the renal proximal tubule cells on the collagen film is about 5×10⁴ cells/cm² to 5×10⁶ cells/cm².

In an embodiment, the diameter of the collagen film is about 6 mm to 10 mm, the thickness is about 120 μm to 200 μm.

In an embodiment, the diameter of the collagen film is about 8 mm, the thickness is about 160 μm, and the density of the renal proximal tubule cells on the collagen film is about 5×10⁵ cells/cm².

In addition, the present application provides a use of the composition of corneal implantation for implanting in patients with damaged corneal endothelial cells. Preferably, the renal proximal tubule cells are autologous renal proximal tubule cells of the patients. Preferably, the use is applied in corneal endothelial transplantation.

Besides, the present application provides a preparation method of the composition of corneal implantation, comprising the following steps:

providing renal proximal tubule cells of a patient,

seeding the renal proximal tubule cells into a cell culture container with a collagen film placed at the bottom, and

cultivating for about 5-10 days to obtain the composition for corneal implantation.

In an embodiment, the renal proximal tubule cells are obtained from the expanded culture of the kidney tissue of the patient.

In an embodiment, about 5×10⁴ of the renal proximal tubule cells are seeded into the cell culture container with the collagen film placed at the bottom, and the density of the cells on the collagen film reaches about 5×10⁵ cells/cm² after cultivating for 7 days.

The details of the invention are set forth in the following description, which is to be regarded as illustrative methods and materials only, and not restrictive. Other similar or equivalent methods and materials described herein to practice or test the present invention should be regarded as the scope of the resent application. In the specification and the appended claims, the singular form includes the plural as well unless the context clearly indicates otherwise. Unless otherwise defined, all technical and scientific terms used herein have the same meaning as generally understood as one having ordinary skill in the art of the present invention.

### BRIEF DESCRIPTION OF THE DRAWINGS

The above and other aspects, features and other advantages of the present invention will be more clearly understood from the following detailed description taken in conjunction with the accompanying drawings, in which:

FIG. 1A shows the result of test 1 of day 0 after implanting.

FIG. 1B shows the result of test 1 of day 7 after implanting.

FIG. 1C shows the result of test 1 of day 30 after implanting.

FIG. 1D shows the result of test 1 of day 60 after implanting.

FIG. 1E shows the result of test 1 of day 90 after implanting.

FIG. 2A shows the result of test 2 of day 0 after implanting.

FIG. 2B shows the result of test 2 of day 3 after implanting.

FIG. 2C shows the result of test 2 of day 7 after implanting.

FIG. 2D shows the result of test 2 of day 30 after implanting.

FIG. 2E shows the result of test 2 of day 60 after implanting.

FIG. 2F shows the result of test 2 of day 90 after implanting.

FIG. 3A shows the result of test 3 of day 0 after implanting.

FIG. 3B shows the result of test 3 of day 3 after implanting.

FIG. 3C shows the result of test 3 of day 7 after implanting.

FIG. 3D shows the result of test 3 of day 30 after implanting.

FIG. 3E shows the result of test 3 of day 60 after implanting.

FIG. 3F shows the result of test 3 of day 90 after implanting.

FIG. 4A shows the result of test 4 of day 0 after implanting.

FIG. 4B shows the result of test 4 of day 3 after implanting.

FIG. 4C shows the result of test 4 of day 7 after implanting.

FIG. 4D shows the result of test 4 of day 30 after implanting.

FIG. 4E shows the result of test 4 of day 60 after implanting.

FIG. 4F shows the result of test 4 of day 90 after implanting.

FIG. 5A shows the ZO-1 staining result of test 1.

FIG. 5B shows the Na—K ATPase staining result of test 1.

FIG. 5C shows the N-cadherin staining result of test 1.

FIG. 5D shows the negative control group of test 1.

FIG. 6A shows the Na—K ATPase staining result of test 2.

FIG. 6B shows the N-cadherin staining result of test 2.

FIG. 6C shows the ZO-1 staining result of test 2.

FIG. 6D shows the GLUT1 staining result of test 2.

### DETAILED DESCRIPTION OF THE PREFERRED EMBODIMENT

Reference will be made in detail description to the exemplary embodiments and drawings for being more readily understood to the advantages and features of the present invention, as well as the methods of attaining them. However, the present invention may be carried out in many different forms and should not be construed as limited to the embodiments set forth herein. Conversely, these embodiments are provided to render the present disclosure to be conveyed the scope of the present invention more thoroughly, completely, and fully to one having ordinary skill in the art of the present invention. Moreover, the present invention would be defined only by the appended claims. As used herein, the term “and/or” includes any and all combinations of one or more of the associated listed components.

Unless otherwise defined, all terms (including technical and scientific terms) used herein have the same meaning as generally understood by one having ordinary skill in the art of the present invention. It will be more understandable that, for example, the terms defined in commonly used dictionaries should be understood to have meanings consistent with the contents of the relevant fields, and would not be interpreted overly idealized or overly formal unless clearly defined herein. As described in the present specification, a range of values is used as a shorthand to describe each and every numerical value in the range, and any number within that range may be chosen as the end-value of that range.

In order to make the disclosed content more concise and easy to understand, the following elements with the same or similar functions will be described with the same symbols, and descriptions of the same or equivalent features will be omitted.

The present invention provides a composition of corneal implantation, comprising: a collagen film, and renal proximal tubule cells, attached to the collagen film.

In an embodiment, the density of the renal proximal tubule cells on the collagen film is about 5×10⁴ cells/cm² to 5×10⁶ cells/cm².

In an embodiment, the diameter of the collagen film is about 6 mm to 10 mm, the thickness is about 120 μm to 200 μm.

In an embodiment, the diameter of the collagen film is about 8 mm, the thickness is about 160 μm, and the density of the renal proximal tubule cells on the collagen film is about 5×10⁵ cells/cm².

In addition, the present invention provides a use of the composition of corneal implantation for implanting in patients with damaged corneal endothelial cells. Preferably, the renal proximal tubule cells are autologous renal proximal tubule cells of the patients. Preferably, the use is applied in corneal endothelial transplantation.

Besides, the present invention provides a preparation method of the composition of corneal implantation, comprising the following steps:

providing renal proximal tubule cells of a patient,

seeding the renal proximal tubule cells into a cell culture container with a collagen film placed at the bottom, and

cultivating for about 5-10 days to obtain the composition for corneal implantation.

In an embodiment, the renal proximal tubule cells are obtained from the expanded culture of the kidney tissue of the patient.

In an embodiment, about 5×10⁴ of the renal proximal tubule cells are seeded into the cell culture container with the collagen film placed at the bottom, and the density of the cells on the collagen film reaches about 5×10⁵ cells/cm² after cultivating for 7 days.

### Example 1—Acquisition of Renal Proximal Tubule Cells

Human specimens were obtained from patients who had kidney tumors and were assessed by doctors to undergo nephrectomy. Without affecting the pathological diagnosis, specimens were collected from the kidney tissue during nephrectomy. The renal cortex was selected and the specimen was cut into the size of one cubic centimeter with the surgical scissor and forceps. 3 pieces of specimens were collected, stored in Hanks' Balanced Salt Solution (HBSS), and placed at 4° C. The specimens for animal experiments were obtained from the autologous kidneys of the animals in the same way.

The specimens were cut into small pieces after washing with PBS, which were put into an Eppendorf then cut into mud with small scissors. The specimens were placed in a 10 cm dish and disintegrated for 30 minutes at 37° C. by 60 mg of collagenase dissolved in 20 ml of Hank's Balanced Salt Solution (HBSS) containing Ca2⁺. After using a syringe to disperse pellets, all the liquid was sucked into a 50 ml centrifuge tube, then 30 ml of HBSS was added in and centrifuged at 1000 rpm for 4 minutes. After removing the supernatant, 10 ml of HBSS was added to disperse pellets and filtered through a 250 μm mesh and centrifuged at 1000 rpm for 4 minutes. The supernatant was removed again, and 30 ml of 45% Percoll solution (Pharamacia Biotech) was added to disperse pellets and centrifuged at 1000 rpm, 4° C., for 30 minutes.

Since the number of renal tubular cell layers is very small, after seeing the cell layering, a drop was taken and observed under the microscope to find the layer where the renal tubules are located. The renal tubule cells were placed in a 15 mL centrifuge tube, 10 mL of HBSS was added, and centrifuged at 800 rpm for 4 minutes. After removing the supernatant, 10 mL of cell culture medium (HPTC) was added to disperse the pellets. The renal proximal tubule cells were seeded in a collagen-coated 10 cm dish and were cultivated an incubator at 37° C., 5% CO₂ for about 10 days to obtain about 7-9×10⁶ of renal proximal tubule cells. In addition, the expanded renal proximal tubule cells can be collected and subcultured with 0.05% of trypsin/EDTA.

### Example 2—Preparation of Composition of Corneal Implantation

A collagen film with a diameter of 8 mm and a thickness of about 160 μm was washed twice with PBS, then placed on the bottom of the 48-well plate with an O-ring pressed above to prevent the film from floating. The renal proximal tubule cells obtained by the method of Example 1 at a number of 5×10⁴ cells were seeded into the wells containing the collagen films, which was cultivated in an incubator at 37° C., 5% CO₂ for 7 days to obtain a collagen film attached with about 5×10⁵ cells/cm² of renal proximal tubule cells.

### Example 3—Animal Experiments

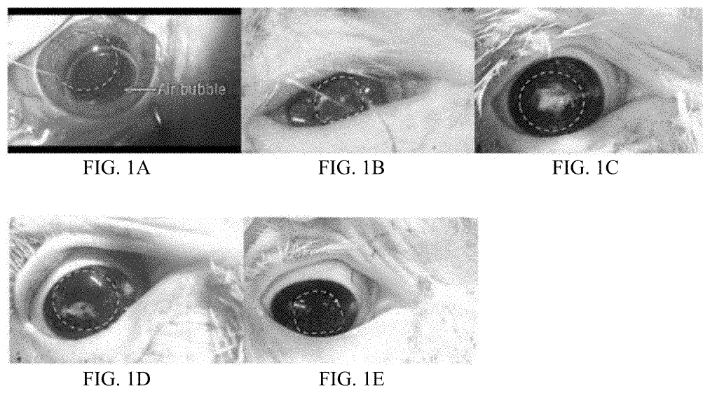

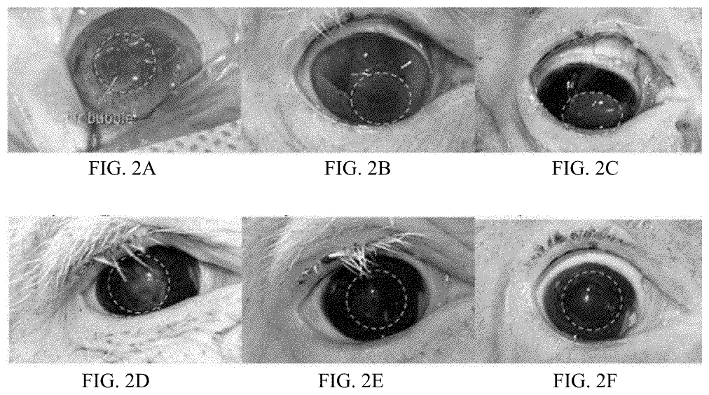

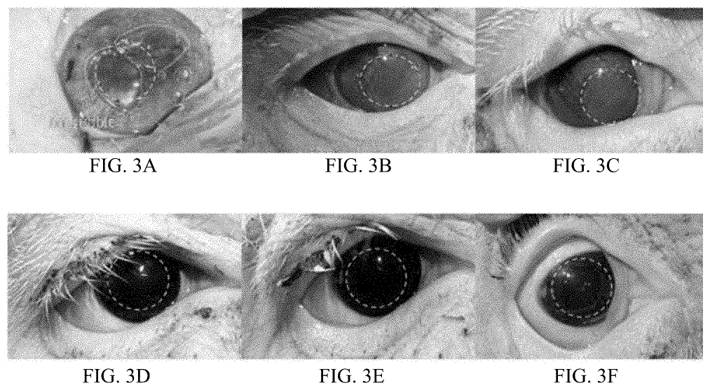

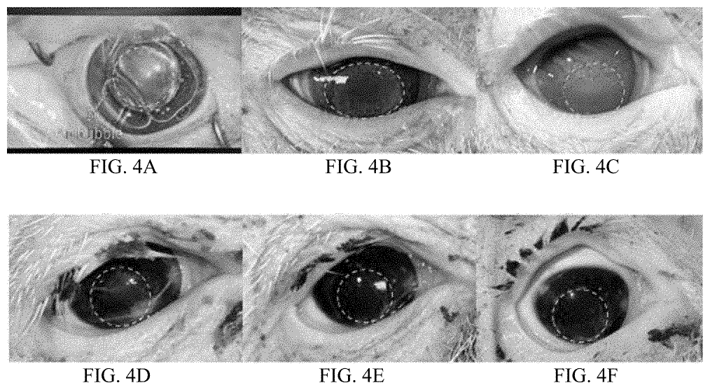

The composition of corneal implantation obtained by the method of Example 2 was implanted into the corneas of pigs by the standard operating method of the Descemet's Stripping Automated Endothelial Keratoplasty (DSAEK). FIGS. 1A-1E to FIGS. 4A-4F show the results of tests 1 to 4 respectively, and the results of tests 1 to 4 are assessed the levels of transparency (1 to 4 points, 1 is the most blur, 4 is the most transparent), redness/swelling (1 to 3 points, 1 is the least red and swollen, 3 is the reddest and the most swollen), and attachment (0 to 2 points, respectively, no attachment, partial attachment, and full attachment) after implantation respectively. The assessment results are summarized as the following Table 1:

TABLE 1

| | Transparency | Redness/Swelling | Attachment |
|---|---|---|---|
| Test 1 | 4 | 1 | 2 |
| Test 2 | 4 | 1 | 2 |
| Test 3 | 4 | 1 | 2 |
| Test 4 | 3 | 1 | 2 |

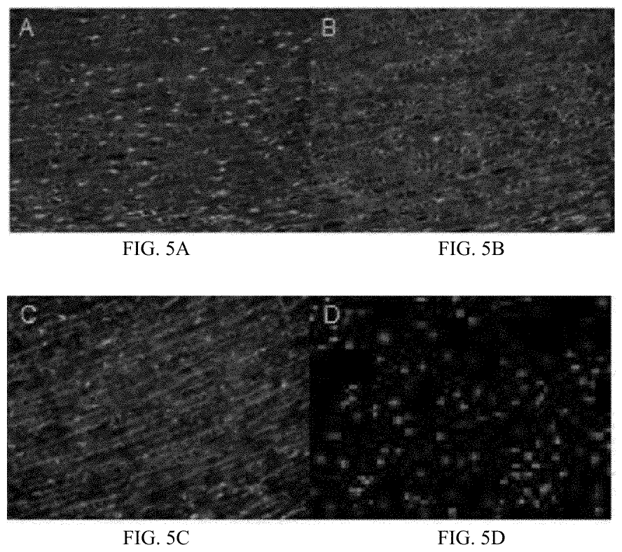

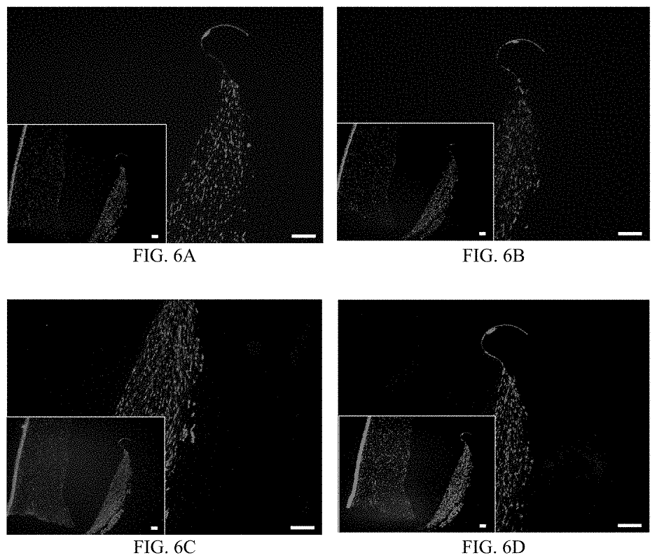

In addition, the frozen sections of the corneas of Experiment 1 were subjected to immunofluorescence staining of ZO-1, Na/K ATPase, and N-cadherin to examine the viability of the renal proximal tubule cells. As shown in FIGS. 5A to 5C, the renal proximal tubule cells were still alive after 120 days of implantation. Besides, the frozen sections of the corneas of Experiment 2 were subjected to immunofluorescence staining of Na/K ATPase, N-cadherin, ZO-1, and GLUT1. As shown in FIGS. 6A-6D, it was also found that the renal proximal tubule cells were still alive after 120 days of implantation.

As can be seen from FIGS. 1A-1E to FIGS. 4A-4F and Table 1, the composition of corneal implantation of the present invention has good transparency, low redness and swelling, and can completely attach after implanting to the cornea. As shown in FIGS. 5A-5D to FIGS. 6A-6D, renal proximal tubule cells attached to the collagen film can survive and be active after implantation. At the same time, the collagen film can strengthen the attachment and will be gradually absorbed after implantation, leaving the renal proximal tubule cells to replace the corneal endothelial cells. Furthermore, there is no rejection problem since the autologous renal proximal tubule cells are used. Therefore, the composition of corneal implantation of the present invention can be used as a medical material of an autologous cell membrane to replace the original damaged corneal endothelial cells, thereby restoring the functionality of the corneal endothelial cells.

What is claimed is:

1. A composition of corneal implantation, comprising:
a collagen film, and
renal proximal tubule cells, attached to the collagen film.

2. The composition of claim 1, wherein the density of the renal proximal tubule cells on the collagen film is about 5×10⁴ cells/cm² to 5×10⁶ cells/cm².

3. The composition of claim 1, wherein the diameter of the collagen film is about 6 mm to 10 mm, the thickness is about 120 μm to 200 μm.

4. The composition of claim 1, wherein the diameter of the collagen film is about 8 mm, the thickness is about 160 μm, and the density of the renal proximal tubule cells on the collagen film is about 5×10⁵ cells/cm².

5. A use of the composition according to claim 1 for implanting in patients with damaged corneal endothelial cells.

6. The use of claim 5, wherein the renal proximal tubule cells are autologous renal proximal tubule cells of the patients.

7. The use of claim 5, wherein the use is applied in corneal endothelial transplantation.

8. The use of claim 5, wherein the density of the renal proximal tubule cells on the collagen film is about 5×10⁴ cells/cm² to 5×10⁶ cells/cm².

9. The use of claim 5, wherein the density of the renal proximal tubule cells on the collagen film is about 5×10⁴ cells/cm² to 5×10⁶ cells/cm².

10. The use of claim 5, wherein the diameter of the collagen film is about 8 mm, the thickness is about 160 μm, and the density of the renal proximal tubule cells on the collagen film is about 5×10⁵ cells/cm².

11. A preparation method of the composition according to claim 1, comprising the following steps:
providing renal proximal tubule cells of a patient,
seeding the renal proximal tubule cells into a cell culture container with a collagen film placed at the bottom, and
cultivating for about 5-10 days to obtain the composition for corneal implantation.

12. The preparation method of claim 11, wherein the renal proximal tubule cells are obtained from the expanded culture of the kidney tissue of the patient.

13. The preparation method of claim 11, wherein about 5×10⁴ of the renal proximal tubule cells are seeded into the cell culture container with the collagen film placed at the bottom, and the density of the cells on the collagen film reaches about 5×10⁵ cells/cm² after cultivating for 7 days.

14. The preparation method of claim 11, wherein the density of the renal proximal tubule cells on the collagen film is about 5×10⁴ cells/cm² to 5×10⁶ cells/cm².

15. The preparation method of claim 11, wherein the diameter of the collagen film is about 6 mm to 10 mm, the thickness is about 120 μm to 200 μm.

16. The preparation method of claim 11, wherein the diameter of the collagen film is about 8 mm, the thickness is about 160 μm, and the density of the renal proximal tubule cells on the collagen film is about 5×10⁵ cells/cm².
